(19) United States
(12) Patent Application Publication
(10) Pub. No.: US 2011/0293530 A1
(43) Pub. Date: Dec. 1, 2011

(54) Methods and Compositions for Using Bleomycin-Derivatized Microbubbles

(75) Inventors: Sidney Hecht, Phoenix, AZ (US); Marek Belohlavek, Scottsdale, AZ (US)

(73) Assignee: ARIZONIA BOARD OF REGENTS, SCOTTSDALE, AZ (US)

(21) Appl. No.: 13/128,142

(22) PCT Filed: Nov. 23, 2009
(86) PCT No.: PCT/US09/65535

§ 371 Date: Aug. 8, 2011

Related U.S. Application Data
(60) Provisional application No. 61/118,285, filed on Nov. 26, 2008.

(51) Int. Cl. A61K 49/00; A61P 35/00

(52) U.S. Cl. 424/9.52

(57) ABSTRACT

Methods and compositions for using tumor targeting compounds bound to microbubbles to facilitate drug delivery and diagnostic imaging at tumor sites.

### RELATED APPLICATIONS

This application claims priority to U.S. Provisional Patent Application Ser. No. 61/118,285 filed Nov. 26, 2008, incorporated by reference herein in its entirety.

### FIELD OF THE INVENTION

The invention relates to imaging and targeting of tumor cells using microbubbles having covalently attached bleomycin.

### BACKGROUND OF THE INVENTION

Ultrasound contrast is a very useful and widely used medical diagnostic technique. The technique takes advantage of the fact that the various fluids and tissues in the body reflect sound waves differently. This results in a contrast between reflected waves that can be detected and used to form an image of the tissue. Ultrasound is used for many different diagnostic purposes, e.g., prenatal imaging or to image blood flow the heart and arteries and observe blockages in blood circulation.

Ultrasound imaging can be greatly enhanced through the use of contrast agents, which when placed in the tissue to be imaged create a greater difference in the reflectance of the sound waves between the tissue to be imaged and the surrounding tissue. This allows much sharper delineation of tissue boundaries and perfusion to be observed.

Such contrast agents are based on the acoustic impedance mismatch between a gas and a liquid. These agents are typically micron-sized bubbles, “microbubbles,” containing various gases encapsulated in polymers, surfactants, proteins, polyaminoacids and their derivatives, liposomes, or inorganic shells.

Typically, the microbubbles are encapsulated to allow a more uniform population of bubble size because unencapsulated gas microbubbles tend to expand and smaller microbubbles tend to diminish in size. Encapsulated contrast agents are well known to those skilled in the art, e.g., U.S. Pat. No. 5,614,169 (describing microbubbles encapsulated with carbohydrates and amphiphilic organic acids); U.S. Pat. No. 5,352,436 (describing microbubbles stabilized by two different surfactants); U.S. Pat. Nos. 4,681,119, 4,442,843 and 4,657,756 which disclose the use of particulate solids having a plurality of gas-filled voids and preferably also a plurality of nuclei for microbubble formation. Other microbubble forming compositions are described in U.S. Pat. No. 4,684,479; U.S. Pat. No. 4,466,442; U.S. Pat. No. 5,573,751; U.S. Pat. No. 5,352,436; U.S. Pat. No. 5,656,211; European Patent Application 0231091 also teaches methods for preparing gas-filled microbubbles.

ALBUNEX® is a contrast agent made of a suspension of stable microencapsulated air bubbles which are encapsulated in human serum albumin. OPTISON® contrast agent is a suspension of stable microencapsulated octafluoropropane bubbles which are encapsulated in human serum albumin. Both are prepared by sonicating dilute human albumin at a temperature slightly below denaturing. ALBUNEX® is prepared by sonicating in the presence of air; OPTISON® in the presence of octafluoropropane. Both are composed of gas-filled microbubbles with a mean diameter in the range of 3-5 microns and stabilized by a thin albumin shell. Levovist® is another commercially available contrast agent that is FDA approved and is made by Schering. It has an air core and a lipid-galactose shell (Lindner, J. R. Nature Rev., 3, 527-532, 2004).

Thus, microbubbles ultrasound contrast agents are made of a shell enclosing a gas core. The shell is usually composed of albumin, galactose or lipids. The make-up of the gas core is extremely important as it defines the ability of the microbubbles to strongly reflect ultrasound waves. Air or heavy insoluble gases such as perfluorocarbons or nitrogen (Lindner, J. R. Nature Rev., 3, 527-532, 2004) are typically used. When microbubbles are administrated intravenously to the systemic circulation, their echogenocity allows contrast-enhanced ultrasound and improved medical sonography. In medical imaging, these agents have applications in radiology and cardiology. See Hamilton, A. J., et al., J. Am. Coll. Cardiol., 43, 453-60, (2004); Christensen, J. P., et al., Circulation, 96, 473-82 (2002).

### BRIEF SUMMARY OF THE INVENTION

In a first aspect, the present invention provides compositions comprising (a) a microbubble comprising an outer shell, wherein the outer shell is derivatized with a first member of a binding pair; and (b) bleomycin bound to the microbubble, wherein the bleomycin is derivatized with a second member of the binding pair, and wherein the first member of the binding pair and the second member of the binding pair are bound to each other. In one embodiment, the bleomycin is selected from the group consisting of bleomycin A2, bleomycin B2, bleomycin A6, and bleomycin A5. In a further embodiment, the first and second members of the binding pair comprise streptavidin and biotin.

In a second aspect, the present invention provides methods of binding bleomycin to a microbubble said method comprising (a) obtaining a microbubble composition, wherein the microbubble composition comprises a first member of a binding pair in its outer shell; and (b) contacting a bleomycin derivative to the microbubble composition, wherein the bleomycin derivative comprises a second member of the binding pair, wherein the contacting occurs under conditions suitable to promote binding of the first member to the second member. In one embodiment, the binding pair members comprise streptavidin and biotin. In another embodiment, the bleomycin is selected from the group consisting of bleomycin A2, bleomycin B2, bleomycin A6, and bleomycin A5. In a further embodiment, the method comprises (a) preparing biotin-N-hydroxysuccinimide ester by treating biotin with 1,1′ carbonyldiimidazole followed by N-hydroxysuccinimide in DMF; (b) incubating the biotin-N-hydroxysuccinimide ester a copper salt of bleomycin in 0.1 M NaOAc for a period sufficient to produce biotinylated CuII•bleomycin; and (c) optionally incubating said biotinylated CuII•bleomycin in a metal ion chelating solution to remove copper ion from the bleomycin to produce biotinylated bleomycin, if removal of the copper ion is desired

In a third aspect, the present invention provides methods of selectively targeting a tumor comprising administering to a subject with a tumor a composition of the invention under conditions suitable to promote binding of the tumor targeting compound to the tumor. In one embodiment, the composition comprises one or more compounds toxic to the tumor, and wherein the method is used to inhibit tumor growth.

In a fourth aspect, the present invention provides methods of selectively imaging a tumor in a patient, comprising administering to a subject with a tumor a composition of the invention under conditions suitable to promote binding of the tumor targeting compound to the tumor; and (b) acquiring an ultrasound image of the composition in the subject. In one embodiment, the method is carried out following a treatment to inhibit tumor growth, and wherein the method is used to monitor effects of the treatment.

### BRIEF DESCRIPTION OF SEVERAL VIEWS OF THE DRAWINGS

FIG. 1 is an illustration of the biotinylated bleomycin A5 ligand with annotation of its biotin component.

FIG. 2 is a schematic representation of the biotinylated bleomycin A5 ligand and its conjugation to the surface of the microbubble.

FIG. 3 shows MCF-7 breast carcinoma control (all but BLM) 400×.

FIG. 4 shows MCF-7 breast carcinoma+BLM biot. 400×.

FIG. 5 shows MCF-10A ‘normal’ breast cells+BLM biot. 400×.

FIG. 6 shows MCF-7 human breast carcinoma cells control (all but BLM) 400×.

FIG. 7 shows MCF-7 human breast carcinoma cells control Blenoxane® 400×.

FIG. 8 MCF-7 human breast carcinoma cells+biot-deglyco-BLM-A₅ 400×.

FIG. 9 shows MCF-10A breast ‘normal” cells control (all but BLM) 400×.

FIG. 10 shows MCF-10A human ‘normal’ breast cells control Blenoxane® 400×.

FIG. 11 shows MCF-10A human ‘normal’ breast cells+biot deglyco-BLM-A₅ 400×.

### DETAILED DESCRIPTION OF THE INVENTION

In a first aspect, the present invention provides compositions comprising

(a) a microbubble comprising an outer shell, wherein the outer shell is derivatized with a first member of a binding pair; and

(b) bleomycin bound to the microbubble, wherein the bleomycin is derivatized with a second member of the binding pair, and wherein the first member of the binding pair and the second member of the binding pair are bound to each other.

In another aspect, the present invention provides methods of binding bleomycin to a microbubble said method comprising (a) obtaining a microbubble composition, wherein the microbubble composition comprises a first member of a binding pair in its outer shell; and (b) contacting a bleomycin derivative to the microbubble composition, wherein the bleomycin derivative comprises a second member of the binding pair, wherein the contacting occurs under conditions suitable to promote binding of the first member to the second member.

As used herein, “bleomycin” (BLM) is a family of structurally related glycopeptide antibiotic compounds, and includes various metallo-BLM compounds, including but not limited to FeIII•BLM CoIII•BLM, ZnII•BLM and CuII•bleomycin. In specific embodiments, the bleomycin comprises or consists of BLM A2, BLM B2, BLM A6, or BLM A5, metallo-versions thereof, or a combination thereof. BLM is produced by the bacterium Streptomyces verticillus, and is commercially available from a variety of sources. BLM is used clinically as an antitumor agent, and is known to localize in many types of tumors, including those against which it has no anti-tumor effect.

In the present invention it is demonstrated for the first time that binding of bleomycin to microbubbles via a binding pair interaction specifically allows tumor imaging with the microbubbles. This finding is useful in providing novel methods of imaging tumors and also for achieving tumor cell-specific targeted delivery of bleomycin as well as additional agents that may be formulated into the bleomycin containing microbubbles. The present invention for the first time provides a description of methods and compositions for using bleomycin containing microbubbles in methods of targeting, diagnosis and treatment of tumor cells, and can be extended to other tumor targeting compounds. The presence of the bleomycin on the microbubbles specifically targets the microbubbles to tumor cells where the microbubbles attach to the tumor cells. As such, this targeted delivery of bleomycin, which can be achieved without the use of ultrasound, allows delivery of the microbubbles and any agent contained therein directly to a tumor site. Thus, in one embodiment, the microbubbles may further comprise one or more further anti-tumor agents which can then be delivered at the tumor site when the microbubble dissipates. Such additional anti-tumor agents can be any suitable for a given purpose, including but not limited to cisplatin, carboplatin, spiroplatin, iproplatin, paclitaxel, docetaxel, rapamycin, tacrolimus, asparaginase, etoposide, teniposide, tamoxifen, amsacrine, mitotane, topotecan, tretinoin, hydroxyurea, procarbazine, BCNU (carmustine) and other nitrosourea compounds, as well as others classified as alkylating agents (e.g., mechlorethamine hydrochloride, cyclophosphamide, ifosfamide, chlorambucil, melphalan, busulfan, thiotepa, carmustine, estramustine, dacarbazine, omustine, streptozocin), plant alkaloids (e.g., vincristine, vinblastine, vinorelbine, vindesine), antimetabolites (e.g., folic acid analogs, methotrexate, fludarabine), pyrimidine analogs (fluorouracil, fluorodeoxyuridine, cytosine arabinoside, cytarabine, azidothymidine, cysteine arabinoside, and azacytidine), purine analogs (mercaptopurine, thioguanine, cladribine, pentostatin, arabinosyl adenine), antitumor antibiotics (e.g., adriamycin, dactinomycin, daunorubicin, doxorubicin, amsacrine, idarubicin, mitoxantrone, bleomycin, plicamycin, ansamitomycin, mitomycin), aminoglutethimide (an aromatase inhibitor), flutamide (an anti-androgen), gemtuzumab ozogamicin (a monoclonal antibody), and oprelvekin (a synthetic interleukin), as well as cell cycle inhibitors and EGF receptor kinase inhibitors in general.

Microbubbles are ultrasound contrast agents made of a shell enclosing a gas core. The shell is usually composed of albumin, galactose or lipids. The make-up of the gas core defines the ability of the microbubbles to strongly reflect ultrasound waves. Air or heavy insoluble gases such as perfluorocarbons or nitrogen (Lindner, J. R. Nature Rev., 3, 527-532, 2004) are typically used. When microbubbles are administrated intravenously to the systemic circulation, their echogenocity allows contrast-enhanced ultrasound and improved medical sonography. In medical imaging, these agents have applications in radiology and cardiology. See Hamilton, A. J., et al., J. Am. Coll. Cardiol., 43, 453-60, (2004); Christensen, J. P., et al., Circulation, 96, 473-82 (2002). Currently, two FDA-approved microbubbles are available. Optison, made by GE Healthcare has an albumin shell and an octofluoropropane gas core, Levovist, made by Schering, had a lipid-galactose shell and air core (Lindner, J. R. Nature Rev., 3, 527-532, 2004).

In one embodiment, the microbubbles have a diameter of about 0.1 to 10 microns. In a further embodiment, the microbubbles have a diameter between 1-4 um, which allows the microbubbles to flow freely through the circulation and microcirculation. Circulation time can be greatly improved by the use of lipid-based membranes coated with longer chain fully saturated lipid molecules, such as distearoylphosphatidylcholine. See Rychak, J. J., et al., J. Con. Rel., 114, 288-99, (2006). Microbubbles designed with low solubility gases such as decafluorobutane slowed the rate at which gas diffused into the bloodstream, thus allowing the microbubble to retain its structure longer. See Klibanov, A. L., Bioconjugate Chem., 16, 9-17, (2005). With these characteristics, contrast agents can be regarded as pure intravascular tracers that behave similarly to red blood cells within the microcirculation and entry into the bloodstream is made possible by simple intravenous insertion via a catheter. See Lindner, J. R., Nature Rev., 3, 527-32, (2004).

The microbubbles are formed by entrapping the gas into a liquid. The microbubbles may be made of various insoluble gases such as fluorocarbon or sulfur hexafluoride gas. The liquid includes any liquid which can form microbubbles. Generally any insoluble gas can be used. It must be gaseous at body temperature and be nontoxic. The gas must also form stable microbubbles of average size of between about 0.1 and 10 microns in diameter when the pharmaceutical composition is sonicated to form microbubbles. Generally perfluorocarbon gases such as perfluoromethane, perfluoroethane, perfluoropropane, perfluorobutane, perfluoropentane are preferred. Other inert gases such as sulfur hexafluoride are also useful in forming microbubbles.

Once the microbubbles are formed they may be stabilized by coating with a suitable lipid of protein, such as albumin, human gamma, globulin, human apotransferrin, Beta lactose and urease.

Microbubbles may be formed by sonication, typically with a sonicating horn. Sonication by ultrasonic energy causes cavitation within the dextrose albumin solution at sites of gas in the fluid. These cavitation sites eventually resonate and produce small microbubbles (about 7 microns in size) which are non-collapsing and stable. In general, sonication conditions which produce concentrations of greater than about 4×10⁸m of between about 5 and about 6 micron microbubbles are preferred. Generally the mixture will be sonicated for about 80 seconds, while being perfused with an insoluble gas. A variety of other methods used to make microbubbles are described in published PCT application WO 96/39197. This same application also describes many of the gases which may be included within the microbubbles. Any such methods can be used to prepare microbubbles to be conjugated to bleomycin analogs as described herein.

Moreover, there are various sources of commercially available microbubbles that can be derivatized with bleomycin as described herein. For example, Optison® (GE HealthCare) and Levovist (Schering). In exemplary embodiments described herein the microbubbles used are Targestar^{B} Ultrasound Contrast Agent was used. To create biotinylated microbubbles, biotin-bleomycin A₅ ligand was conjugated to Targestar^{B} Targeted Ultrasound Contrast Agent (Targeson). Four hundred microliters of Coupling Reagent (Targeson) were added to 1.5 mL of conjugated Targestar^{B} microbubbles and incubated for 20 min at room temperature, with gentle agitation every 5 min. The product was divided into two syringes, rinsed with 1.75 mL of Infusion Buffer, and then centrifuged for 3 min at 400×g (10° C.). The infranatant was then drained to 1 mL. Fifty μL of biotinylated bleomycin A₅ was added to one of the vials and both were incubated at room temperature for 20 min with gentle agitation every 5 min. To each sample was added 1.75 mL of Infusion Buffer before centrifugation for 3 min at 400×g (10° C.). This solution was then drained to 1.0 mL before recovery of the supernatant and repetition of the previous step. Finally, the supernatant was resuspended in Infusion Buffer to a final volume of 2.0 mL.

Any suitable binding pair can be used to for binding bleomycin to the microbubble. Preferably, the binding pair is one with a dissociation constant of 10⁻³M or less; in other embodiments, a dissociation constant of 10⁻⁴M or less; 10⁻⁵M or less; or 10⁻⁶M or less. In one embodiment, the binding pair comprises biotin-streptavidin. Other non-limiting embodiments include metal/chelators binding pairs; protein/protein binding pairs; protein-cofactors binding pairs; (modified) nucleic acid-nucleic acid binding pairs; and protein/nucleic acid binding pairs. Any suitable method can be used to derivatize the microbubble and the bleomycin to incorporate the binding pair member into their structure; examples of such methods are provided below. In one embodiment, the method comprises the method comprises (a) preparing biotin-N-hydroxysuccinimide ester by treating biotin with 1,1′ carbonyldiimidazole followed by N-hydroxysuccinimide in DMF; (b) incubating the biotin-N-hydroxysuccinimide ester a copper salt of bleomycin in 0.1M NaOAc for a period sufficient to produce biotinylated CuII•bleomycin; and (c) optionally incubating said biotinylated CuII•bleomycin in a metal ion chelating solution to remove copper ion from the bleomycin to produce biotinylated bleomycin, if removal of the copper ion is desired. As will be understood by those of skill in the art, other metallo-BLM forms can be obtained in this method by admixture of the desired metal ion in stoichiometric amount to the metal free bleomycin.

In a third aspect, the present invention provides methods of selectively targeting a tumor comprising administering to a subject with a tumor a composition of the invention under conditions suitable to promote binding of the tumor targeting compound to the tumor. In one embodiment, the composition comprises one or more compounds toxic to the tumor, and wherein the method is used to inhibit tumor growth. In one embodiment of the method, the composition comprises one or more further anti-tumor compounds (such as those described above), and the method is used to inhibit tumor growth. In this embodiment, bleomycin serves to target the composition to the tumor, and the one or more further anti-tumor compounds is used to inhibit tumor growth. Any tumor type that bleomycin localizes to (including, but not limited to, carcinomas breast, lung, prostate, skin, brain, kidney, and colon) can be treated using the methods of the invention, by appropriate use of compositions of the invention comprising further anti-tumor compounds.

In a further aspect, the present invention provides methods of selectively imaging a tumor in a patient, comprising administering to a subject with a tumor a composition of the invention under conditions suitable to promote binding of the tumor targeting compound to the tumor; and (b) acquiring an ultrasound image of the composition in the subject. In one embodiment, the method is carried out following a treatment to inhibit tumor growth, and wherein the method is used to monitor effects of the treatment. Since bleomycin specifically targets the microbubbles to tumor cells, the mass of the tumor can be monitored diagnostically. For example, the tumor cells can be monitored before during and after therapy to determine the efficacy of the therapy and to verify delivery of the therapeutic to the tumor. Any tumor type that bleomycin localizes to (including, but not limited to, carcinomas breast, lung, prostate, skin, brain, kidney, and colon) can be imaged using the methods of the invention.

In these methods of the invention, once the bleomycin-containing microbubbles are prepared they are delivered to a subject in need thereof. The method preferred for practicing the delivery of the microbubble composition involves obtaining a composition comprising the bleomycin-containing microbubbles of the invention; introducing the composition agent into the subject by intravenous injection, intravenously (i.v. infusion), percutaneously or intramuscularly. The microbubble is then processed in the subject. The presence of the bleomycin or bleomycin analog covalently linked to the outer surface of the microbubble specifically targets the microbubble to tumor cells within the subject. At the tumor site, the bubble eventually dissipates delivering the bleomycin (as well as any additional agent containing in the bubble) at the site of the tumor cells.

Once prepared, the conjugated microbubbles are transferred to a sterile syringe and injected parenterally into a subject (for example, a mammal), preferably near the target site of activity of the agent. The microbubbles prepared according to examples presented below can be used to attach specifically to tumor cells in any subject. The compositions are administered using conventional methods for delivering such compositions, i.e., using parenteral administration of microbubbles, preferably at or near the site of the tumor. The imaging of the tumor site is then achieved through conventional methods that involve imaging of diagnostic contrast agents.

Unless the context dictates otherwise, all embodiments of one aspect of the invention are suitable for use with other aspects of the invention, and the different embodiments can be combined.

The following examples are for illustration purposes only and are not intended to limit this invention in any way. It will be appreciated by those of skill in the art that numerous other protein-bioactive agent combinations can be used in the invention and are even contemplated herein. In all the following examples, all parts and percentages are by weight unless otherwise mentioned, all dilutions are by volume.

### Example 1

### Surface Biotinylation with Bleomycin A₅

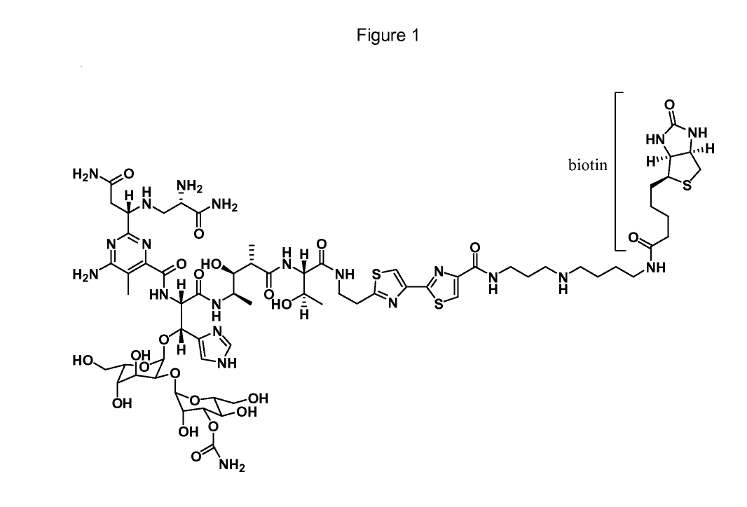

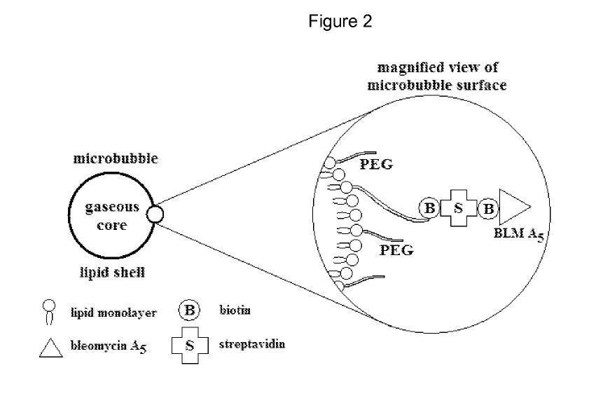

One method of conjugating bleomycin A₅ to the surface of a microbubble is to use a biotin-streptavidin complex. By biotinylating bleomycin, it is possible to achieve non-covalent attachment with streptavidin. This biotin-streptavidin linkage can then modulated by the presence of a protective polymer spacer, such as polyethylene glycol (PEG), which is on the surface of the microbubble agent. To insure batch consistency in the creation of microbubbles and therefore stability and size during experimentation, pre-synthesized microbubbles were used in all experimental procedures. In particular, the product Targestar^{B} Ultrasound Contrast Agent (Targeson, Charlottesville, Va.) was used because it is designed with a biotin site modulated with a PEG spacer to allow for rapid conjugation to the modified bleomycin A₅ ligand (FIG. 1). These microbubbles have a consistent mean diameter size of ˜2.5 μm, a mean number of 1.5×10⁶ streptavidin molecules/microbubble (per Targeson protocol #P09-02), and a concentration of about 1×10⁸ microbubbles/mL (according to Protocol #1002-Targestar^{B} Ligand Conjugation).

Although microbubbles were originally designed for ultrasound imaging, they may also be viewed using a parallel plate flow chamber and an inverted microscope. See Klibanov, A. L., et al. Contrast Media Mol. Imaging, 1, 259-66, (2006); Talkalkar, A. M., et al., J. Con. Rel., 96, 473-82, (2004).

Experimental Procedures:

Preparation of Biotin N-Hydroxysuccinimide Ester (2) To a solution of 101 mg (0.41 mmol) of biotin in 2 mL of DMF was added 67 mg (0.41 mmol) of 1,1′-carbonyldiimidazole at 78° C. with further heating until CO₂ evolution ceased. The solution was cooled to room temperature and stirred for 3 h. To the reaction mixture was added a solution of 47 mg (0.41 mmol) of N-hydroxysuccinimide in 2 mL of DMF. The solution was stirred overnight at room temperature. The solvent was concentrated under diminished pressure and the product was crystallized from 2-propanol and then DMF-ether to afford a fine white powder yield 95 mg (68%). ¹H NMR (CDCl₃) δ 1.43-1.66 (m, 7H), 2.58 (m, 1H), 2.66 (t, 2H), 2.75 (s, 4H), 3.05 (m, 1H), 4.11 (m, 1H), 4.27 (m, 1H), 6.36 (d, 2H, J=18.4 Hz); mass spectrum, m/z 342.1 (M⁺).

Preparation of Biotinylated BLM A₅ (4) To a solution of 10 mg (7.0 μmol) of Cu(II)•BLM A₅ in 0.75 mL of a 0.1 M NaOAc_{(aq)} solution at 0° C. was added 7.0 mg (21.0 μmol) of 2. The solution was stirred at 0-4° C. for 48 h. The solution was treated with 3 aliquots of 5 mL of CHCl₃ to extract unreacted 2. The solution was lyophilized to afford a blue powder. The solution was dissolved in a 15% EDTA solution and stirred overnight at room temperature. The crude product was applied to an Alltima C₁₈ column (8×1 cm) and washed with 200 mL of H₂O. The product was eluted with a 4:1 MeOH-2 mM AcOH solution. The fractions containing the product were co-evaporated with toluene to remove trace amounts of AcOH. The product suspended in H₂O was lyophilized to afford a white powder. The resulting powder was purified further by reversed phase HPLC on an Alltima C₁₈ column to afford the final product, biotinylated bleomycin A₅; yield 3.2 mg (30%); mass spectrum, m/z 1667.6 (M)⁺.

Preparation of Biotinylated DeglycoBLM A₅ (8). To 10 mg (9.3 μmol) of deglycoBLM A₅ (5) in 1 mL of H₂O was added 1.3 mg (9.3 μmol) of CuCl₂ at room temperature. The water was removed by lyophilization. The residue was dissolved in 3.0 mL of 0.1 M NaOAc solution and treated with 10 mg (0.027 mmol) of biotin ester 2 at 0° C. The solution was stirred at 0-4° C. for 48 h at which time the solution was extracted with three 5-mL portions of CHCl₃ to remove unreacted 2. The aqueous solution was lyophilized to afford a fine blue powder. The powder was dissolved in a 15% EDTA solution and stirred overnight at room temperature. The solution was lyophilized and the resulting powder was purified by reversed phase HPLC on an Alltima C₁₈ column: yield 0.3 mg (3%); m/z, 1322.0 (M)⁺.

Biological Preparation

MCF-7 human breast carcinoma cells (ATCC, Manassas, Va.) were grown on sterile glass cover slips (40 mm) and incubated at 37° C. until they reached 40-60% confluency in a 5% fetal bovine serum/RPMI1640 medium solution.

Biotinylated bleomycin A₅ was weighed and dissolved in 500 μL Infusion Buffer (Targeson). The UV absorbance of this solution was measured using a SpectraMax M5 UV/Vis spectrophotometer (Molecular Devices, Sunnyvale, Calif.) at 284 nm. The concentration of the solution was then calculated using Beer-Lambert's Law, A=εbc, using the values c=1 cm for the path length of the quartz cuvette used and ε=14,500 M⁻¹cm⁻¹ for the molar absorptivity of bleomycin, as defined by Manderville, R. A., Ellena, J. F., Hecht, S. M., J. Am. Chem. Soc. 117, 7891-903, (1995).

To create biotinylated microbubbles, the synthesized biotin-bleomycin A₅ ligand was conjugated to Targestar^{B} Targeted Ultrasound Contrast Agent (Targeson). Four hundred microliters of Coupling Reagent (Targeson) were added to 1.5 mL of conjugated Targestar^{B} microbubbles and incubated for 20 min at room temperature, with gentle agitation every 5 min. The product was divided into two syringes, rinsed with 1.75 mL of Infusion Buffer, and then centrifuged for 3 min at 400×g (10° C.). The infranatant was then drained to 1 mL. Fifty μL of biotinylated bleomycin A₅ was added to one of the vials and both were incubated at room temperature for 20 min with gentle agitation every 5 min. To each sample was added 1.75 mL of Infusion Buffer before centrifugation for 3 min at 400×g (10° C.). This solution was then drained to 1.0 mL before recovery of the supernatant and repetition of the previous step. Finally, the supernatant was resuspended in Infusion Buffer to a final volume of 2.0 mL.

Attachment of the biotinylated microbubble to the cultured MCF-7 breast cancer cells was imaged using an inverted microscope Zeiss Axiovert 200M fitted with an AxioCam MRm camera. Adherent cancer cells on glass cover slips were assembled into a parallel plate flow chamber (Bioptechs FCS2, Micro-Environmental Systems) with a constant temperature maintained at 37° C. The prepared solution of biotinylated microbubbles was introduced into the parallel plate flow chamber via ⅛-inch diameter tubing (Silastic) at a controlled rate of 0.01 mL/min using an adjustable infusion-withdrawal syringe pump (Harvard Apparatus, Holliston, Mass.). A negative control experiment was performed using a preparation of Targestar^{B} microbubbles without any added biotin-bleomycin A₅ ligand. ‘Normal’ human breast MCF-10A cells were used as well for comparison purposes.

When using other cell lines, the exact media and growing conditions have been outlined in Table 2. All cell lines were grown to 40-60% confluency.

### Example 2

### Selective Attachment and Targeting of Breast Cancer Cells

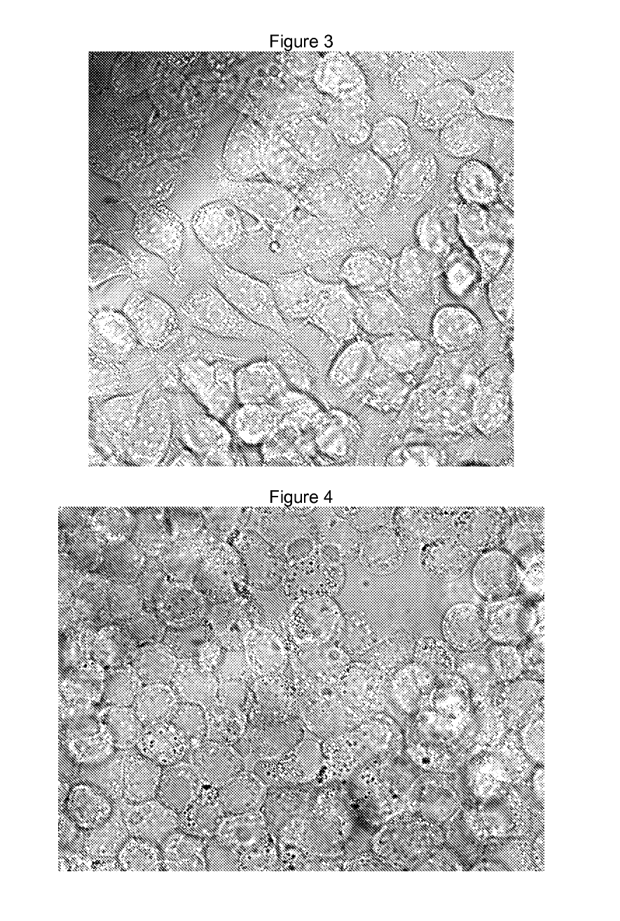

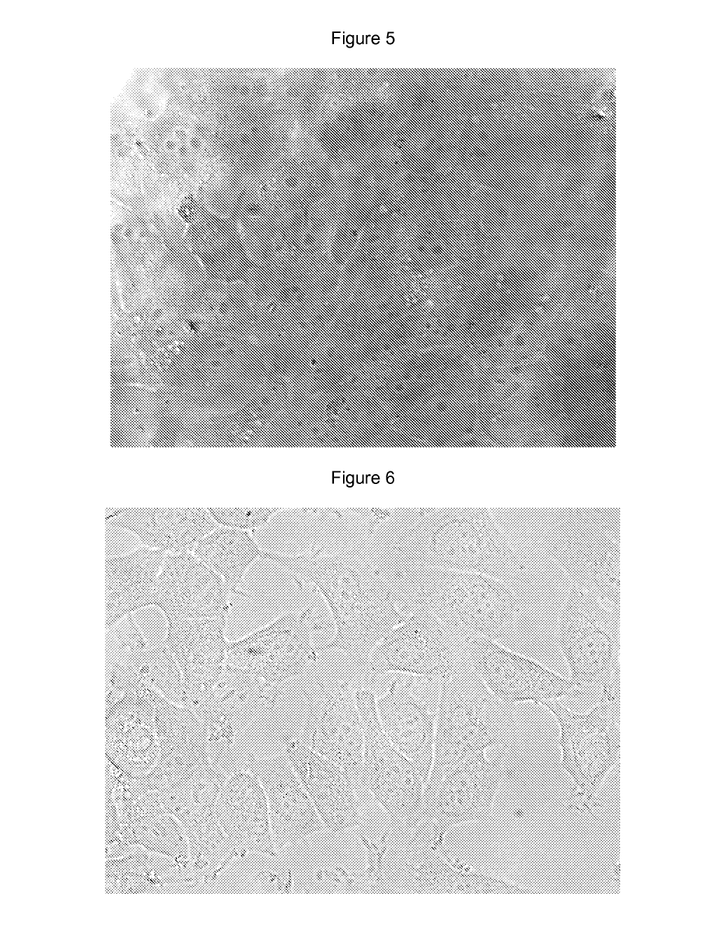

Reviewing the data shown in FIGS. 3, 4 and 5 it can be seen that bleomycin containing microbubbles selectively targeted to breast cancer cells. These results confirmed the conjugation of bleomycin A₅ to the microbubbles as well as the selective attachment of the conjugate to the MCF-7 human breast carcinoma cells (FIG. 4), compared to a control containing all components except the biotinylated BLM A₅ (FIG. 3). Biotinylated BLM A₅ attachment was not observed when an identical experiment was conducted using MCF-10A ‘normal’ breast cells (FIG. 5). These data show that the bleomycin covalently linked to microbubbles specifically attaches to cancer cells and does not attach to normal cells. Such selective attachment of bleomycin to the cancer cells could form the basis for diagnostic imaging at tumor sites.

Similar experiments were conducted using the same concentrations of deglyco BLM A₅. FIGS. 6 to 11 demonstrate that biotinylated deglycobleomycin A₅ does not promote Targestar B microbubble attachment to the human breast cancer or ‘normal’ cells, while an equimolar concentration of biotinylated bleomycin did promote attachment to the same cancer cells.

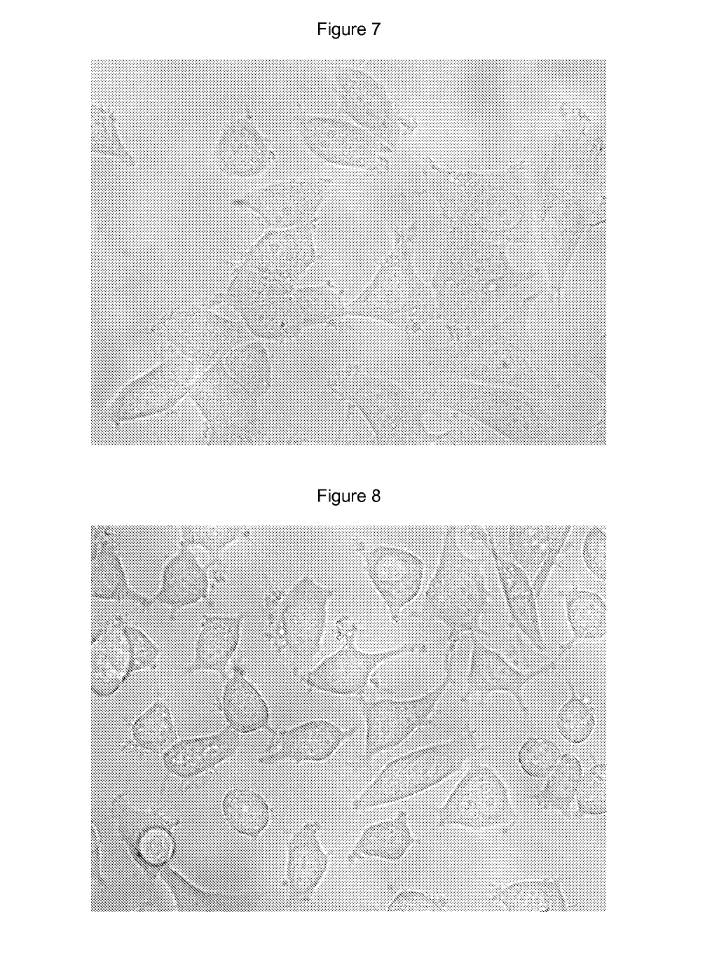

In FIG. 6 it can be seen that there was no attachment of the microbubbles to the cancer cells in the absence of Blenoxane® (bleomycin sulfate; primarily a mixture of bleomycin congeners A₂ and B₂; not biotinylated) or biotinylated bleomycin A₅. Furthermore, when Blenoxane® alone was used (no biotinylation), there still was no evidence of attachment to the cancer cells (FIG. 7). FIG. 8 did not reveal the presence of microbubbles attached to the MCF-7 human breast carcinoma cells when biotinylated deglycobleomycin A₅ was employed.

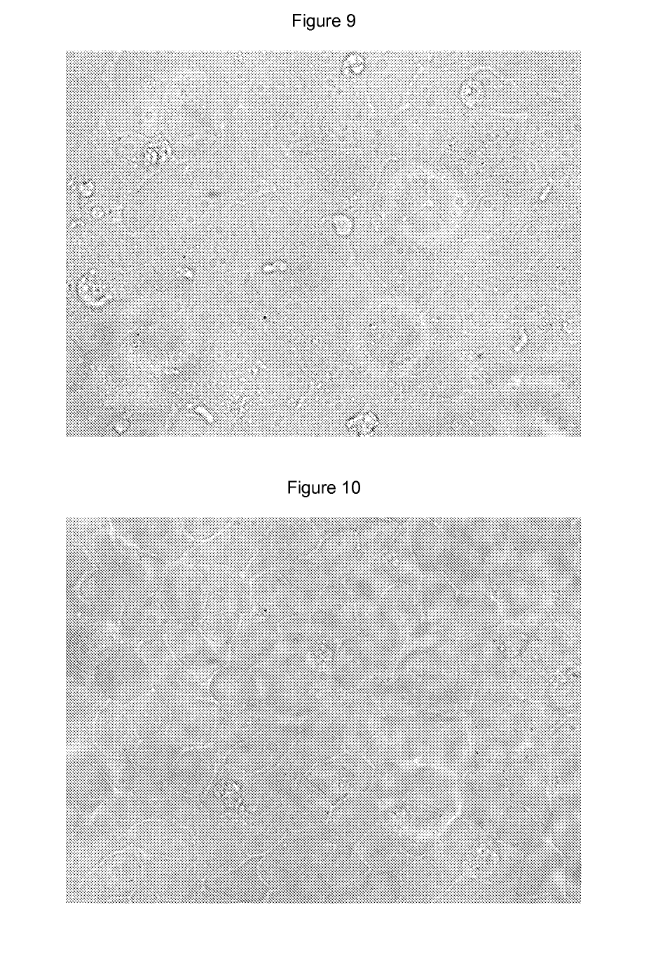

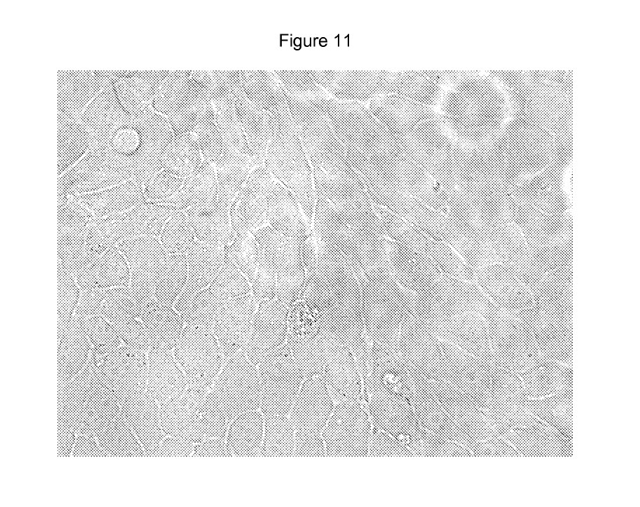

No attachment was observed when an identical experiment was conducted using MCF-10A ‘normal’ breast cells instead of MCF-7 cells (see FIGS. 9 to 11). For example, FIG. 9 shows there was no attachment of the microbubbles to the normal cells (MCF-10A) in the absence of Blenoxane® or biotinylated bleomycin A₅. FIG. 10 shows there was no presence of attached microbubbles when Blenoxane® was used (no biotinylation) in the presence of MCF-10A cells. FIG. 11 shows lack of attachment of microbubbles to MCF-10A human ‘normal’ breast cells in the presence of biotinylated deglycobleomycin A5. Thus, these data demonstrate that biotinylated deglycobleomycin A5 does not promote Targestar B microbubble attachment to the human breast cancer or ‘normal’ cells, while an equimolar concentration of biotinylated bleomycin did promote attachment to the same cancer cells.

Parallel Plate Flow Chamber: Each sample was then run through a parallel plate flow chamber (Bioptechs FCS2, Micro-Environmental Systems) containing human breast carcinoma MCF7 cells. The temperature of the chamber was maintained at 37° C. during the experiment. The flow rate was controlled at a rate of 0.01 mL/min (Harvard Apparatus Syringe Pump 33) and pictures were taken using a Zeiss Axiovert 200M inverted microscope.

Synthesis of the biotinylated BLM to be used in the ultrasound imaging studies is concise (Scheme 1). Biotin was treated with 1,1′-carbonyldiimidazole followed by N-hydroxysuccinimide in DMF to obtain 2 in 68% yield. CuII•BLM A₅ was treated with 2 in a 0.1 M aqueous NaOAc and was stirred for two days to afford Cu bound 4. The biotinylated CuII•BLM was treated with a 15% EDTA solution to remove the metal ion. Removal of the copper was successful as verified by HPLC. The synthesis of biotinylated deglycoBLM A₅ was carried out analogously starting from deglycoBLM A₅ (Scheme 2).

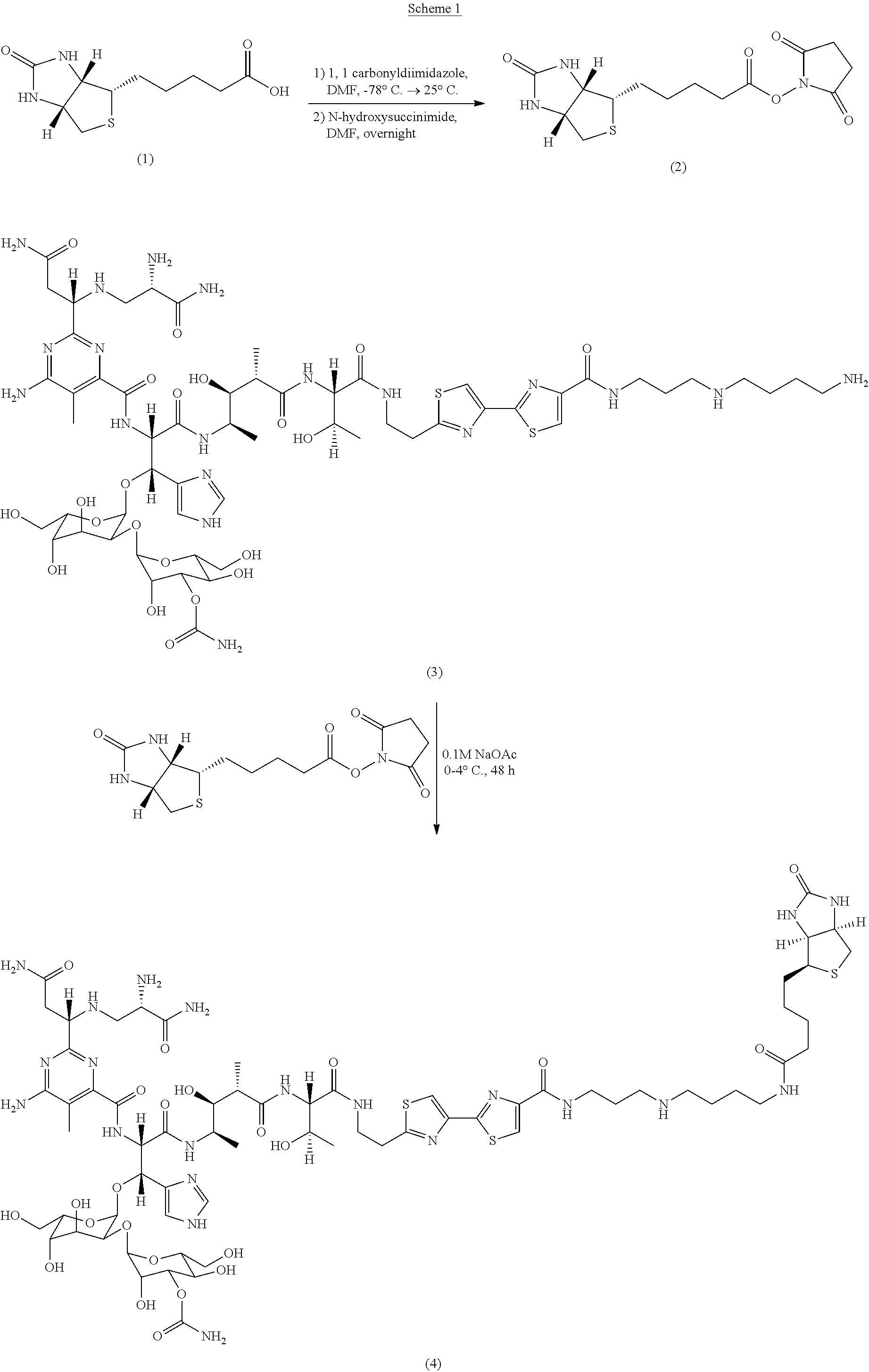

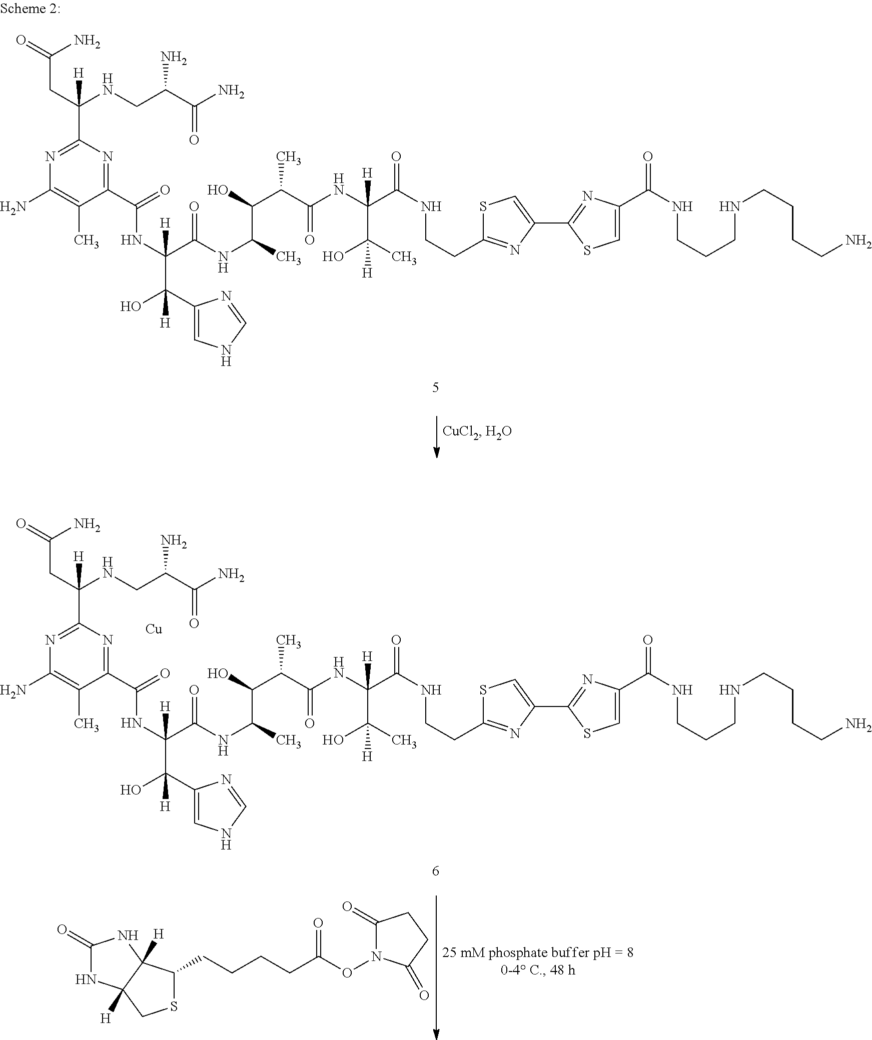

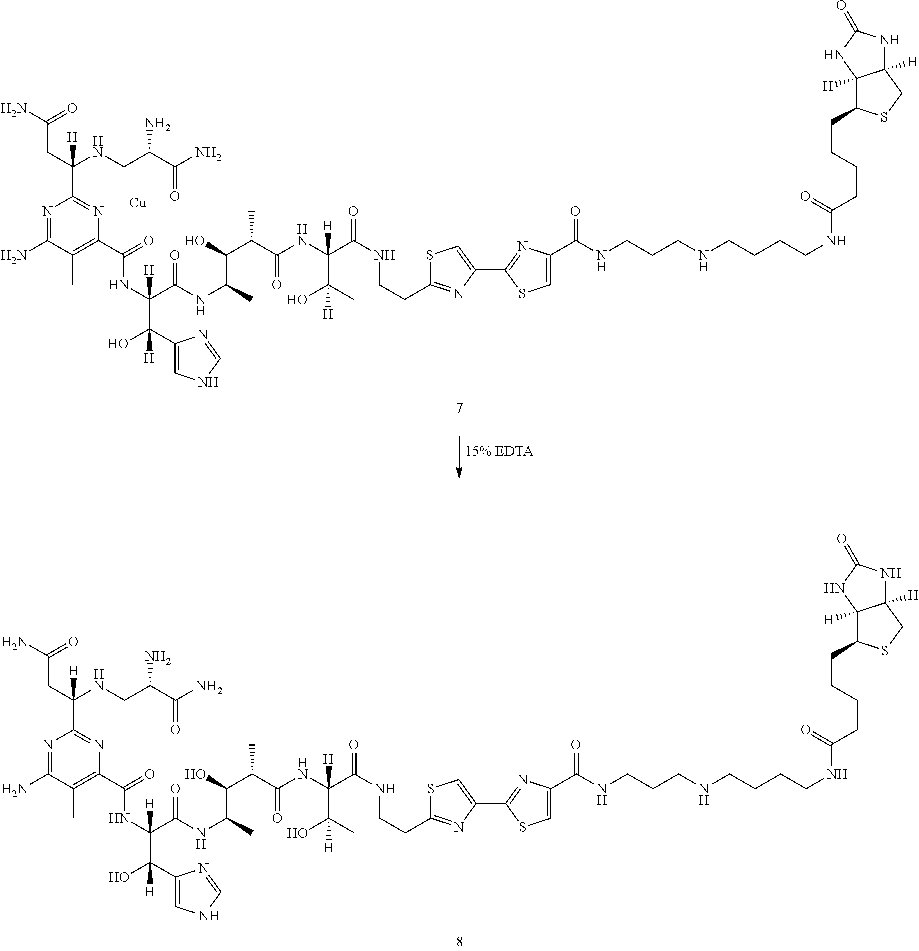

Further experiments were carried out on different tumor cells; the data is summarized in Table 1; information on cell lines used is provided in Table 2:

TABLE 1

| | MCF-7 | MCF-10A | SW-480 | CRL-1541 | DU-145 | CRL-2221 | A-549 | CCL-75 |
|---|---|---|---|---|---|---|---|---|
| | Breast | Normal | Colon | Normal | Prostate | Normal | Lung | Normal |
| | carcinoma | breast | carcinoma | colon | carcinoma | prostate | carcinoma | lung |
| Microbubble + | ++ | − | ++ | − | ++ | − or + | ++ | − |
| Biot. BLM A₅ | | | | | | | | |
| Microbubble + | − | − | − | − | − | − | − | − |
| Blenoxane ® | | | | | | | | |
| Microbubble | − | − | − | − | − | − | − | − |
| only | | | | | | | | |

| | | | | | HTB-12 | | | |
|---|---|---|---|---|---|---|---|---|
| | CRL-7637 | CRL-7636 | CRL-7622 | CCL-211 | Brain | CCL-204 | A-498 | CRL-2305 |
| | Skin | Normal | Bone | Normal | astro- | Normal | Kidney | Normal |
| | melanoma | skin | carcinoma | bone | cytoma | brain | carcinoma | kidney |
| Microbubble + | + | − | − | − | + | − | + | − or + |
| Biot. BLM A₂ | | | | | | | | |
| Microbubble + | − | − | − | − | − | − | − | − |
| Blenoxane ® | | | | | | | | |
| Microbubble | − | − | − | − | − | − | − | − |
| only | | | | | | | | |

(−) denotes no attachment; (+) denotes low positive attachment; (++) denotes high positive attachment

TABLE 2

| Cell | | Tumor/Normal and Metastatic | Growth Conditions (Medium, |
|---|---|---|---|
| Lines | Organs | Sites | Supplements, Atmosphere) |
| MCF-7 | Breast | Breast adenocarcinoma from | RPMI-1640, 10% FBS, 5% CO₂ |
| | | pleural effusion | |
| MCF- | Breast | Mammary gland epithelial | MEBM Bullet Kit, 100 ng/mL |
| 10A | | | cholera toxin, 10% FBS |
| DU-145 | Prostate | Prostate carcinoma from brain | EMEM, 10% FBS, 5% CO₂ |
| CRL- | Prostate | Prostate epithelial transformed | RPMI-1640, 10% FBS, 5% CO₂ |
| 2221 | | with HPV-18 | |
| A-498 | Kidney | Kidney carcinoma | EMEM, 10% FBS, 5% CO₂ |
| CRL- | Kidney | Kidney cells transformed with | RPMI-1640, 10% FBS, 5% CO₂ |
| 2305 | | HPV-16 | |
| A-549 | Lung | Lung carcinoma | RPMI-1640, 10% FBS, 5% CO₂ |
| CCL-75 | Lung | Normal lung fibroblast | EMEM, 10% FBS, 5% CO₂ |
| SW- | Colon | Colorectal adenocarcinoma | Leibovitz's L-15, 10% FBS, 100% |
| 480 | | | air |
| CRL- | Colon | Normal colon fibroblast | EMEM, 10% FBS, 5% CO₂ |
| 1541 | | | |
| HTB-12 | Brain | Brain astrocytoma | Leibovitz's L-15, 10% FBS, 5% CO₂ |
| CCL- | Brain | Normal lung fibroblast from | EMEM, 10% FBS, 5% CO₂ |
| 204 | | patient with astrocytoma | |
| CRL- | Bone | Bone osteosarcoma from lung | DMEM (high glucose), 10% FBS, |
| 7622 | | | 5% CO₂ |
| CCL- | Bone | Normal lung fibroblast from | DMEM (1X glucose), 10% FBS, 5% |
| 211 | | patient with osteosarcoma | CO₂ |
| CRL- | Skin | Skin melanoma from lung | DMEM (high glucose), 10% FBS, |
| 7637 | | | 5% CO₂ |
| CRL- | Skin | Normal skin fibroblast | DMEM (high glucose), 10% FBS, |
| 7636 | | | 5% CO₂ |
| BxPC-3 | Pancreas | Pancreas adenocarcinoma | RPMI-1640, 10% FBS, 5% CO₂ |

Of the seven further cell lines tested (besides MCF-7 and MCF-10A), four cell lines showed complete tumor cell selectivity (SW-480 colon, A549 lung, CRL-7637 skin, and HTB-12 brain). Two cell lines pairs exhibited positive attachment in the ‘normal’ counterparts. However, in both cases, the ‘normal’ cell lines were transformed with Human Papillomavirus (HPV) strains. For instance, ‘normal’ CRL-2221 prostate cells were transformed with HPV-18, and ‘normal’ CRL-2305 kidney cells were transformed with HPV-16. Hence, the positive attachment result can be attributed to the viral transformation of these cells. In the case of CRL-7622/CCL-211 cell pair (bone), no attachment was seen at all. This may be due to the fact that bleomycin usually affects soft cell carcinomas, and thus its effect could be limited when it comes to osteons. Despite the results in the HPV-transformed cells, there was still greater attachment in the tumor counterpart as opposed to its ‘normal’ complement.

From the foregoing, it is apparent that such methods of binding of bleomycin analogues to ultrasound contrast agents, microbubbles, can improve tumor imaging and provide an experimental platform to better understand the targeting behavior of bleomycin.

We claim:

1. A composition comprising
(a) a microbubble comprising an outer shell, wherein the outer shell is derivatized with a first member of a binding pair; and
(b) bleomycin bound to the microbubble, wherein the bleomycin is derivatized with a second member of the binding pair, and wherein the first member of the binding pair and the second member of the binding pair are bound to each other.

2. The composition of claim 1, wherein the bleomycin is selected from the group consisting of bleomycin A2, bleomycin B2, bleomycin A6 and bleomycin A5.

3. The composition of claim 1, wherein the first and second members of the binding pair comprise streptavidin and biotin.

4. The composition of claim 1, wherein the microbubbles have a diameter of between about 0.1 microns to about 10 microns.

5. The composition of claim 1, wherein the microbubbles have a diameter of between about 1 microns to about 4 microns

6. A methods for selectively targeting a tumor comprising administering to a subject with a tumor a composition according to claim 1 under conditions suitable to promote binding of the bleomycin to the tumor.

7. The method of claim 6, wherein the composition further comprises one or more further compounds toxic to the tumor, wherein the method is used to inhibit tumor growth.

8. The method of claim 6, wherein the tumor is selected from the group consisting of carcinomas of the breast, lung, prostate, skin, brain, kidney, and colon.

9. The method of claim 6, wherein the composition is administered intravenously, percutaneously, parenterally, or intramuscularly.

10. A method for selectively imaging a tumor in a patient, comprising
(a) administering to a subject with a tumor a composition according to claim 1 under conditions suitable to promote binding of the bleomycin to the tumor; and
(b) acquiring an ultrasound image of the composition in the subject.

11. The method of claim 10, wherein the method is carried out following a treatment to inhibit tumor growth, wherein the method is used to monitor effects of the treatment.

12. The method of claim 10, wherein the tumor is selected from the group consisting of carcinomas of the breast, lung, prostate, and colon.

13. The method of claim 10, wherein the composition is administered intravenously, percutaneously, parenterally, or intramuscularly.

14. A method for binding bleomycin to a microbubble said method comprising
(a) obtaining a microbubble composition, wherein the microbubble composition comprises a first member of a binding pair in its outer shell; and
(b) contacting a bleomycin derivative to the microbubble composition, wherein the bleomycin derivative comprises a second member of the binding pair, wherein the contacting occurs under conditions suitable to promote binding of the first member to the second member.

15. The method of claim 14, wherein the bleomycin is selected from the group consisting of bleomycin A2, bleomycin B2, bleomycin A6 and bleomycin A5.

16. The method of claim 14 wherein the binding pair members comprise streptavidin and biotin.

17. The method of claim 14, wherein the method comprises
(a) preparing biotin-N-hydroxysuccinimide ester by treating biotin with 1,1′ carbonyldiimidazole followed by N-hydroxysuccinimide in DMF; and
(b) incubating the biotin-N-hydroxysuccinimide ester a copper salt of bleomycin in 0.1M NaOAc for a period sufficient to produce biotinylated CuII•bleomycin.

18. The method of claim 17, further comprising incubating said biotinylated CuII•bleomycin in a metal ion chelating solution.
